(19) United States
(12) Patent Application Publication
(10) Pub. No.: US 2011/0152650 A1
(43) Pub. Date: Jun. 23, 2011

(54) ADAPTIVE PUMP CONTROL DURING NON-INVASIVE BLOOD PRESSURE MEASUREMENT

(75) Inventors: Robert F. Donehoo, Milwaukee, WI (US); Richard Medero, Tampa, FL (US)

(73) Assignee: GENERAL ELECTRIC COMPANY, Schenectady, NY (US)

(21) Appl. No.: 12/643,212

(22) Filed: Dec. 21, 2009

(51) Int. Cl. A61B 5/1455; A61B 5/021

(52) U.S. Cl. 600/324; 600/490

(57) ABSTRACT

A method of operating a non-invasive blood pressure (NIBP) monitor having a blood pressure cuff. During operation of the NIBP monitor, the blood pressure cuff is initially inflated at a rapid inflation rate. Once the blood pressure cuff reaches a first pressure, the inflation rate of the blood pressure cuff is reduced from the rapid inflation rate to a measurement inflation rate. The blood pressure cuff continues to inflate at the measurement inflation rate while the NIBP monitor receives signals from the patient. Based upon the signals received from the patient, the controller of the NIBP monitor calculates an initial inflation pressure. The blood pressure cuff is inflated to the calculated initial inflation pressure and inflation is terminated. In this manner, signals received from the patient during inflation are used to calculate the initial inflation pressure to reduce the amount of time required to make a blood pressure measurement.

### BACKGROUND OF THE INVENTION

The present disclosure generally relates to a method of controlling a blood pressure cuff inflation to enhance the performance of a non-invasive blood pressure (NIBP) system. More particularly, the present disclosure relates to a method of varying the rate of the inflation of the blood pressure cuff to enhance the measurement of a patient blood pressure.

The oscillometric method of measuring blood pressure involves applying an inflatable cuff around an extremity of a patient's body, such as a patient's upper arm. During the use of a conventional non-invasive blood pressure (NIBP) monitoring system, the cuff is inflated to an initial inflation pressure, which is slightly above the patient's systolic pressure. The cuff is then progressively deflated and a pressure transducer detects the cuff pressure, along with pressure fluctuations or oscillations resulting from the beat-to-beat pressure changes in the artery under the cuff. The data from the pressure transducer is used to compute the patient's systolic pressure, mean arterial pressure (MAP) and diastolic pressure. As can be understood, the selection of the initial inflation pressure is an important factor in determining the amount of time required by the NIBP system to measure cuff pressure and to detect cuff oscillations for the estimation of blood pressure.

One requirement in determining the blood pressure using an NIBP monitoring system is that the cuff needs to be inflated above the systolic pressure so that a good representation of the oscillation amplitude pattern can be measured. If a recent blood pressure has already been measured, the systolic information from that previous determination can be used to estimate the initial inflation pressure for the present determination. However, this technique cannot be used if the last determination is not recent, or the patient has been changed, or the instrument has just been powered on. In other words, the determination must be done with no a priori knowledge of an estimate of the blood pressure.

Without any information about the patient, the initial inflation pressure may not be optimal for the particular circumstances being measured. In order to handle this, the system must pump up to a high pressure to guarantee that the initial inflation pressure is above systolic for the patient. Alternatively, the system must, upon observing the oscillation pattern during the deflation, decide that there is not enough information at the high cuff pressure end of the measured oscillometric data to reasonably estimate systolic; this requires further pumping and searching. These scenarios waste time and cause discomfort for the patient.

Thus, if the initial inflation pressure is selected well above the systolic blood pressure for the patient, the NIBP system over inflates the blood pressure cuff, resulting in patient discomfort and extended measurement time. Alternatively, if the initial inflation pressure is selected below the systolic blood pressure for the patient, the blood pressure cuff must re-inflate to obtain an accurate reading. Therefore, it is desirable to have some knowledge of the patient's blood pressure in order to control the cuff inflation and deflation to enhance the performance of an NIBP system.

As can be understood, the selection of the initial inflation pressure determines the amount of time required before the NIBP system begins to deflate the cuff pressure for the purpose of measuring cuff pressure along with detecting cuff pressure oscillations to estimate the patient's blood pressure. When monitoring a patient without any prior measurement information, the system must select an initial inflation pressure. It is desirable for the system to estimate at least the systolic pressure for the patient to enhance the determination of the initial inflation pressure.

### BRIEF DESCRIPTION OF THE INVENTION

The present disclosure relates to a method and system for monitoring the blood pressure in a patient that varies the rate of inflation of a blood pressure cuff to improve the performance of a non-invasive blood pressure (NIBP) monitor. The NIBP monitor includes a blood pressure cuff that is placed on the limb of a patient, such as the arm. The blood pressure cuff is selectively inflated and deflated by a central controller that controls the rate of inflation and deflation of the cuff during the monitoring process.

In one embodiment of the disclosure, the central controller initially inflates the blood pressure cuff at a rapid inflation rate. The blood pressure cuff is inflated to a first pressure at the rapid inflation rate to decrease the amount of time required for the overall blood pressure measurement cycle.

Once the cuff pressure reaches the first pressure, the controller reduces the rate of inflation of the blood pressure cuff to a measurement inflation rate. The controller inflates the blood pressure cuff at the measurement inflation rate while monitoring for signals related to the patient.

In a first embodiment, the signals related to the patient are generated from a pulse monitor. Specifically, the controller of the NIBP monitor receives a plethysmograph signal from the pulse monitor with the heart rate sensor placed on the same limb as the blood pressure cuff. As the blood pressure cuff begins to occlude the artery positioned beneath the blood pressure cuff, the heart rate signals from the pulse monitor change. Based upon the changing signals from the pulse monitor, the controller calculates an initial inflation pressure. The controller continues to inflate the blood pressure cuff to the initial inflation pressure.

Once the blood pressure cuff reaches the initial inflation pressure, the controller begins to deflate the blood pressure cuff in a series of pressure steps in a conventional manner.

In an alternate embodiment, the controller detects oscillation pulses from the blood pressure cuff during the initial inflation of the blood pressure cuff at the measurement inflation rate. Based upon the oscillation pulses detected during the initial inflation, the controller estimates a systolic pressure for the patient. From the estimated systolic pressure, the controller determines an initial inflation pressure and continues to inflate the blood pressure cuff at the measurement inflation rate to the initial inflation pressure.

Once the blood pressure cuff reaches the initial inflation pressure, the controller decreases the pressure within the blood pressure cuff in the series of pressure steps, as is known.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a block diagram of a patient being monitored by an NIBP monitor using an air compressor to inflate the blood pressure cuff;

FIG. 1a is a block diagram of a patient being monitored by an NIBP monitor that inflates the blood pressure cuff using a supply of pressurized air;

FIG. 2 is a graph depicting a standard method of operating an NIBP monitor by detecting two oscillation pulse amplitudes at each of a series of pressure steps during deflation from an initial inflation pressure;

FIG. 3 illustrates the method of varying the inflation rate of a blood pressure cuff and estimating an initial inflation pressure during the inflation of the cuff at a measurement inflation rate;

FIG. 4 is a block diagram of a second embodiment of an NIBP monitoring system for monitoring blood pressure in a patient;

FIG. 5 illustrates the method of varying the rate of inflation of a blood pressure cuff to determine an initial inflation pressure during the inflation process; and

FIG. 6 is a flow chart illustrating the operational sequence used by the system and method of the present disclosure to determine the blood pressure of a patient using an NIBP monitor.

### DETAILED DESCRIPTION OF THE INVENTION

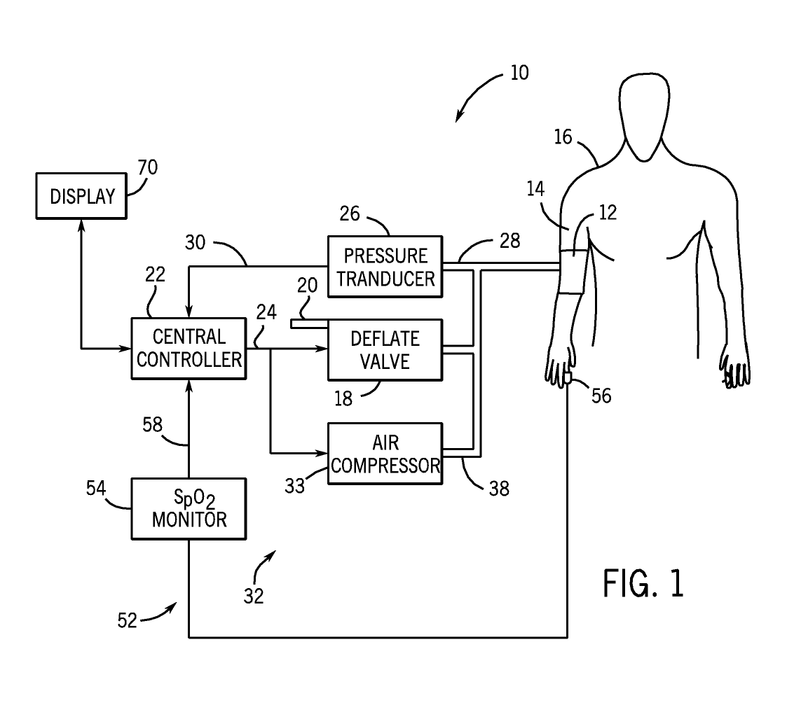

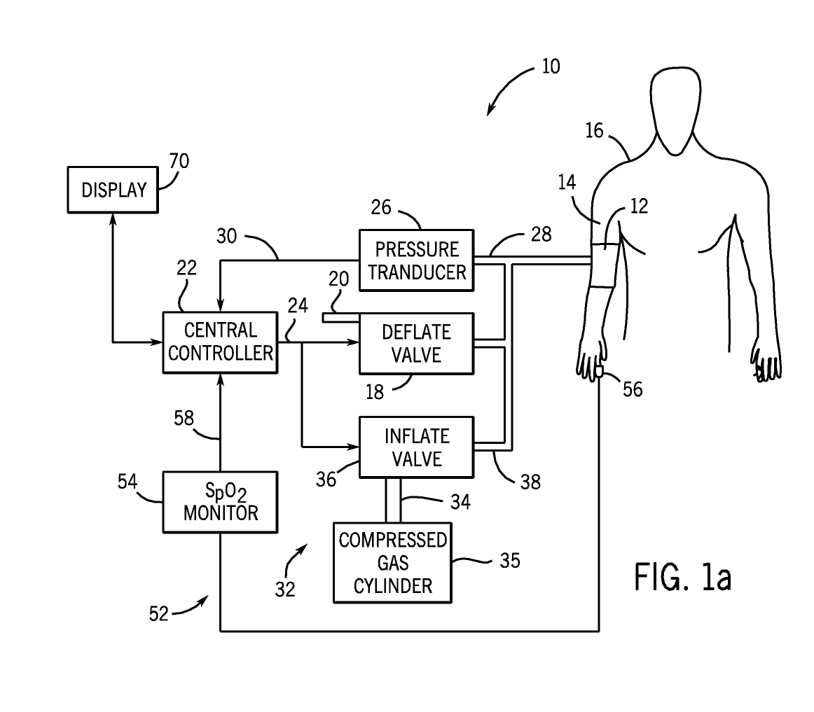

FIG. 1 generally illustrates a non-invasive blood pressure (NIBP) monitoring system 10. The NIBP monitoring system 10 includes a blood pressure cuff 12 placed on the arm 14 of a patient 16. The blood pressure cuff 12 can be inflated and deflated for occluding the brachial artery of the patient 16 when in the fully inflated condition. As the blood pressure cuff 12 is deflated using the deflate valve 18 having exhaust 20, the arterial occlusion is gradually relieved. The deflation of the blood pressure cuff 12 by the deflate valve 18 is controlled by a central controller 22 through the control line 24.

A pressure transducer 26 is coupled by duct 28 to the blood pressure cuff 12 for sensing the pressure within the cuff 12. In accordance with conventional oscillometric techniques, the pressure transducer 26 is used to sense pressure oscillations in the cuff 12 that are generated by pressure changes in the brachial artery under the cuff. The electrical oscillation pulses from the pressure transducer 26 are obtained by the central controller 22, using an analog- to digital converter, through connection line 30.

In FIG. 1a, a source of pressurized air 32, such as an air compressor 33, is connected by duct 34. In the embodiment incorporating an air compressor, the air compressor is coupled directly to the duct 38 and the pressure of gas from the air compressor 33 is variable and controlled by the controller 22. However, if the source of pressurized air 32 is supplied by a compressed gas cylinder 35, as shown in FIG. 1a, an inflate valve 36 is positioned between the compressed gas cylinder 35 and the duct 38. The operation of the inflate valve 36 is controlled by the central controller 22 through the control line 24. Thus, the inflation and deflation of the blood pressure cuff 12 is controlled by the central controller 22 through the deflate valve 18 and the inflate valve 36, respectively.

From the standpoint of the principles of the present invention, the processing of the oscillation signals from first pressure transducer 26 by the central controller 22 to produce blood pressure data, and optionally to reject artifact data, can be conducted in accordance with the prior art teachings of the Ramsey U.S. Pat. Nos. 4,360,029 and 4,394,034. In any event, it is desirable to use any of the known techniques to determine the quality of the oscillation complexes received at each cuff pressure so that the blood pressure determination is made using the physiological relevant cuff pressure oscillations from each heartbeat and not artifacts.

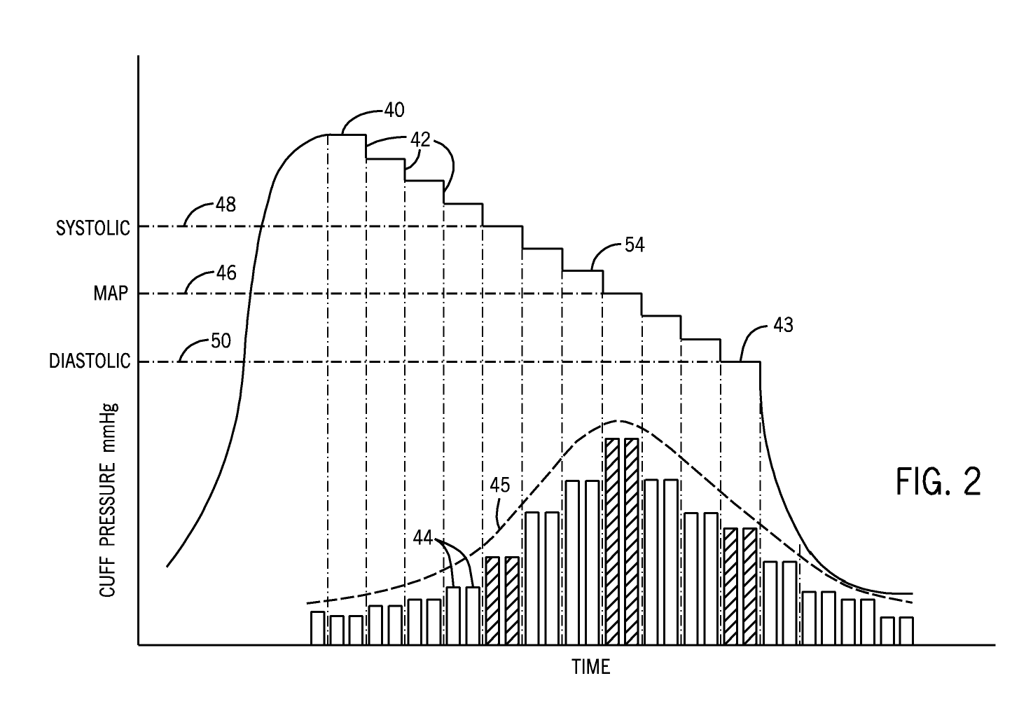

During normal operation of the NIBP monitoring system 10 shown in FIG. 1, the blood pressure cuff 12 is initially placed on the patient 16, typically around the subject's upper arm 14 over the brachial artery. At the inception of the measuring cycle, the blood pressure cuff 12 is inflated to a target inflation pressure that fully occludes the brachial artery, i.e., prevents blood from flowing through the brachial artery at any time in the heart cycle. In FIG. 2, the target inflation pressure is illustrated by reference number 40.

After the blood pressure cuff has been inflated to the target inflation pressure 40, the deflate valve is actuated by the controller to deflate the cuff in a series of pressure steps 42. Although various values for each pressure step 42 can be utilized, in an exemplary example, each pressure step 42 is typically about 8 mmHg per step.

After each pressure step 42, the NIBP monitoring system detects and records the amplitude 44 of two cuff oscillation pulses for the current cuff pressure level. The pressure transducer measures the internal cuff pressure and provides an analog signal characterizing the blood pressure oscillatory complexes. The peak values of the complex signals are determined within the central controller.

As the cuff pressure decreases from the initial inflation pressure, the NIBP monitoring system detects the cuff pressure oscillations 44 and records the pressure oscillation amplitudes for the current cuff pressure. The central controller within the NIBP monitoring system can then calculate the MAP 46, systolic pressure 48 and diastolic pressure 50.

As the measurement cycles progress, the peak amplitude of the oscillation pulses generally become monotonically larger to a maximum and then become monotonically smaller as the cuff pressure continues toward full deflation, as illustrated by the bell-shaped graph 45 in FIG. 2. The peak amplitude of the cuff pressure oscillation complexes, and the corresponding occluding-cuff pressure values, are retained in the central processor memory. The oscillometric measurements are used by the central processor to calculate the mean arterial pressure (MAP) 46, the systolic pressure 48 and the diastolic pressure 50 in a known manner. The calculated blood pressure measurements are viewable on the display 70 shown in FIG. 1.

Referring back to FIG. 1, the system of the first embodiment further includes a pulse monitor 52 for detecting pulse signals from the patient indicative of the patient's heartbeat. In the embodiment of the invention illustrated in FIG. 1, the pulse monitor 52 is a pulse oximeter monitoring system 54 having a sensor that detects a plethysmographic signal from the patient, such as a finger probe 56 positioned on the patient 16 to determine the SpO₂ level of the patient 16.

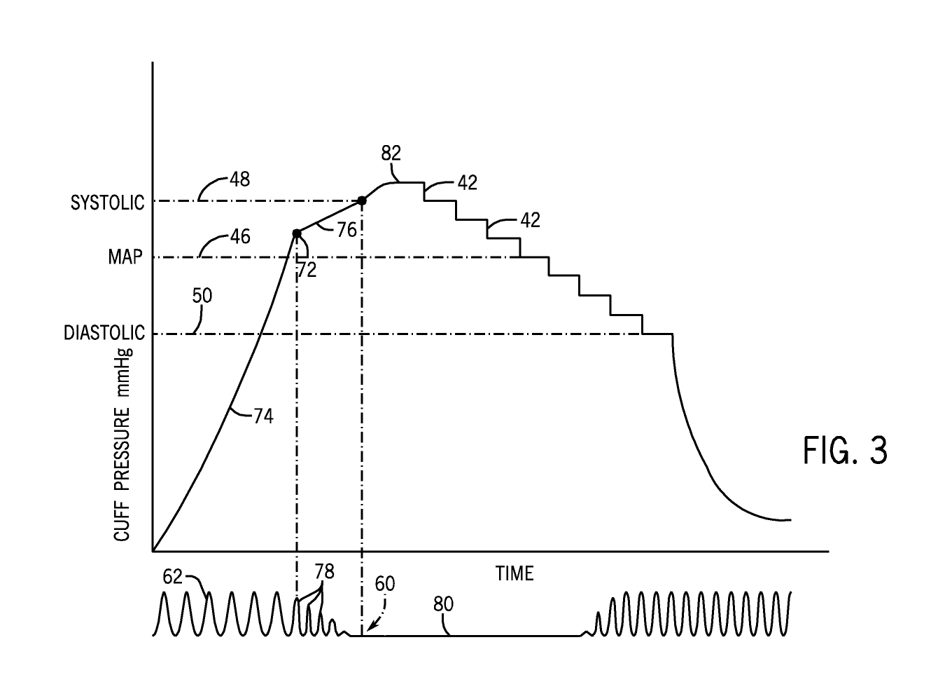

The pulse oximeter monitoring system 54 generates an SpO₂ plethysmographic signal that is provided to the controller 22 of the NIBP monitoring system 10 through a communication line 58. In addition to providing the SpO₂ level for the patient, the pulse oximeter monitor 54 provides a plethysmographic waveform 60 (FIG. 3) that includes a series of pulses 62 that each result from a beat of the patient's heart. Since the finger probe 56 is attached to the patient 16 at all times, the pulse oximeter monitor 54 continuously monitors the patient and generates a continuous plethysmographic waveform 60 having the series of time-spaced pulses 62.

Although a pulse oximeter monitor 54 is shown and described in the embodiment of FIG. 1, it should be understood that other types of pulse monitoring systems and sensors can be utilized while operating within the scope of the disclosure. As an example, an impedance plethysmograph monitor can be placed on the finger or wrist, a piezoelectric sensor could be utilized on the wrist of the patient or any other means of sensing the blood volume pulse within the patient and distal to the blood pressure cuff can be utilized while operating within the scope of the present disclosure.

Referring now to FIG. 3, prior to beginning operation of the NIBP monitoring system to determine the patient blood pressure, the pulse sensor within the finger probe detects a series of individual pulses 62 that each result from a beat of the patient's heart. The continuous plethysmograph signal 60 from the finger probe is obtained by the SpO₂ monitor 54 and relayed to the central controller 22 of the NIBP monitoring system 10, as illustrated in FIG. 1.

Referring now to FIG. 3, when the NIBP monitoring system of the first embodiment begins operation, the blood pressure cuff 12 positioned on the arm of the patient is inflated from approximately 0 mmHg to a first pressure 72 at a very rapid inflation rate, illustrated by the portion of the curve 74 extending from approximately 0 mmHg cuff pressure to the first pressure 72. In the embodiments shown in FIGS. 1 and 1a, the source of pressurized air 32 can be one of two contemplated sources.

One contemplated source is a pressurized gas cylinder 35 (FIG. 1a) that supplies pressurized air to the cuff 12 through the inflation valve 36. The controller 22 provides control signals to the inflation valve 36 through the control line 24. In this manner, controller 22 operates the inflation valve 36 to inflate the pressure cuff 12 at the rapid inflation rate shown by curve 74 in FIG. 3. In one embodiment of the present disclosure, the rapid inflation rate can be 50 mmHg/sec, although other inflation rates are contemplated as being within the scope of the present disclosure.

In a second embodiment of the disclosure, the source of pressurized air 32 can be an air compressor 33 (FIG. 1) that can be operated by the controller to supply pressurized air at various rates. In such an embodiment, the controller provides a control signal to the air compressor to inflate the blood pressure cuff at the rapid inflation rate shown by curve 74.

Referring back to FIG. 3, the controller inflates the blood pressure cuff at the rapid inflation rate until a change is identified in the plethysmographic pulses 62 as they diminish in size, as identified at point 72. As shown in FIG. 3, the pressure point 72 is slightly below the systolic pressure 48 for the patient.

In the embodiments shown in FIG. 1, the system rapidly inflates the blood pressure cuff according to curve 74 from approximately 0 mmHg to a pressure between MAP and systolic. The inflation time from the beginning of the inflation cycle to the first pressure 72 will take approximately 5-7 seconds for an adult blood pressure cuff.

During the rapid inflation of the blood pressure cuff illustrated by curve 74, the controller may receive only a few pulses 62 from the pulse monitor, as illustrated by the plethysmographic wave form 60. As an example, if the patient's heart rate is 50 bpm, only 3-4 heart beats will occur during the rapid inflation. If the blood pressure cuff were inflated at the rapid inflation rate from the first pressure 72 to an initial inflation pressure above the systolic pressure for the patient, the rapid inflation rate would allow only a very few heart beats to be monitored. Therefore, in accordance with the present disclosure, the controller 22 operates the inflate valve 36 or the air compressor 33 to reduce the inflation rate to a measurement inflation rate illustrated by curve 76 shown in FIG. 3. The measurement inflation rate illustrated by curve 76 is well below the rapid inflation rate shown in curve 74. In one illustrated example, the measurement inflation rate is approximately 10 mmHg/second, although other inflation rates are contemplated. However, the measurement inflation rate is well below the rapid inflation rate. During the inflation at the measurement inflation rate, the controller can monitor the plurality of individual pulses 62 that are received from the pulse monitor.

Since the blood pressure cuff 12 and the finger probe 56 are positioned on the same arm of the patient, as the pressure within the blood pressure cuff increases near and above the systolic pressure for the patient, the amplitude of the pulse signals 62 begins to decrease, as shown by the attenuated pulses 78 in FIG. 3. Once the pressure within the blood pressure cuff exceeds the systolic pressure for the patient, the blood flow through the brachial artery past the blood pressure cuff is terminated such that the pulse signals are no longer present in the plethysmographic signal 60, as illustrated by the flat portion 80 of the plethysmographic signal 60.

During operation of the NIBP monitor, the controller 22 receives the heart rate signal from the pulse monitor 54 and can detect the beginning of the attenuated pulse signals 78. Based upon the attenuated pulse signals, the controller can determine an estimated systolic pressure for the patient as the blood pressure cuff is being inflated.

Once the controller calculates the estimated systolic pressure, the controller then calculates an initial inflation pressure 82 that is slightly above the estimated systolic pressure. Preferably, the initial inflation pressure 82 is selected slightly above the estimated systolic pressure such that the blood pressure cut is adequately inflated above the actual systolic pressure 48 for the patient, but yet not significantly above the systolic pressure to avoid patient discomfort and optimize the amount of time required to calculate the blood pressure for the patient.

In addition to estimating the systolic pressure based upon the attenuated pulses 78, the controller could alternatively terminate the inflation of the blood pressure cuff when the amplitude of the attenuated signal falls a selected percentage below the amplitude of the standard pulse signal 62. Further, the decision to terminate the inflation of the blood pressure cuff could also be based upon the rate of change of the baseline signal during inflation of the blood pressure cuff. Although the decision to stop the inflation of the blood pressure cuff could be based upon an amplitude measurement of the pulse signal and the rate of change of the base line signal, it is also contemplated that other pulse parameters could be utilized while operating within the scope of the present disclosure.

Once the blood pressure cuff has been inflated to the initial inflation pressure, the pressure within the blood pressure cuff is deflated in the series of pressure steps 42 and the oscillation pulse amplitudes monitored, as was described with reference to FIG. 2.

As can be understood in the embodiment shown in FIG. 3, the NIBP monitoring system 10 is operated to inflate the blood pressure cuff 12 at a first, rapid inflation rate until the blood pressure cuff reaches a first pressure 72. Once the blood pressure cuff reaches this first pressure, the blood pressure cuff is inflated at a second, measurement inflation rate. During the inflation of the blood pressure cuff at the second measurement inflation rate, the controller monitors the signal from the pulse monitor. Based upon the monitored pulse signal from the pulse monitor, the controller generates an initial inflation pressure 82. The controller allows the blood pressure cuff to be inflated at the measurement inflation rate to the initial inflation pressure 82, where the inflation terminates and the blood pressure cuff is then deflated in a known manner and the blood pressure calculated. The use of the rapid inflation rate to initially bring the blood pressure cuff to the first pressure 72 and the second, reduced measurement inflation rate to monitor the patient during inflation allows the NIBP monitoring system 10 to optimize the amount of time required to determine the patient's blood pressure.

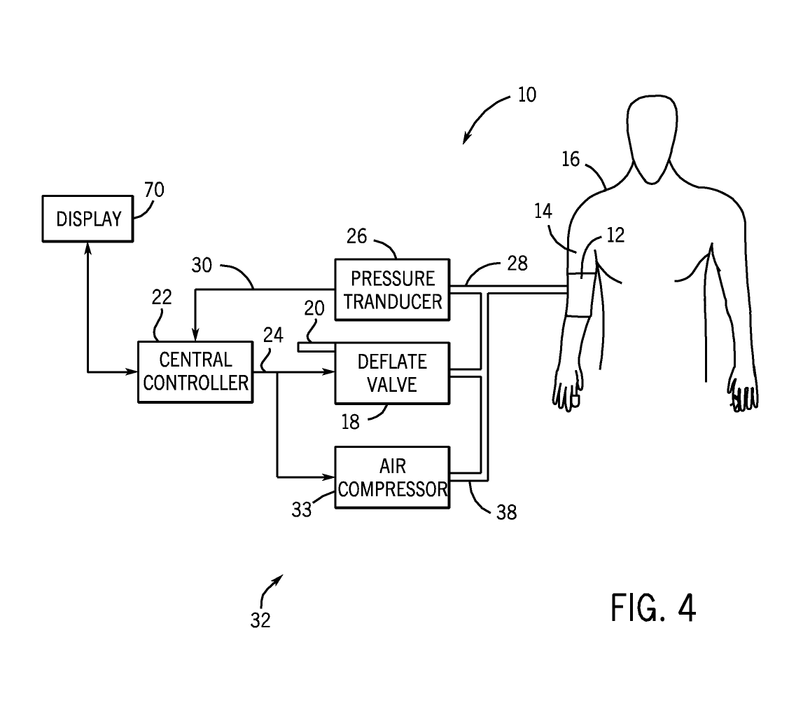

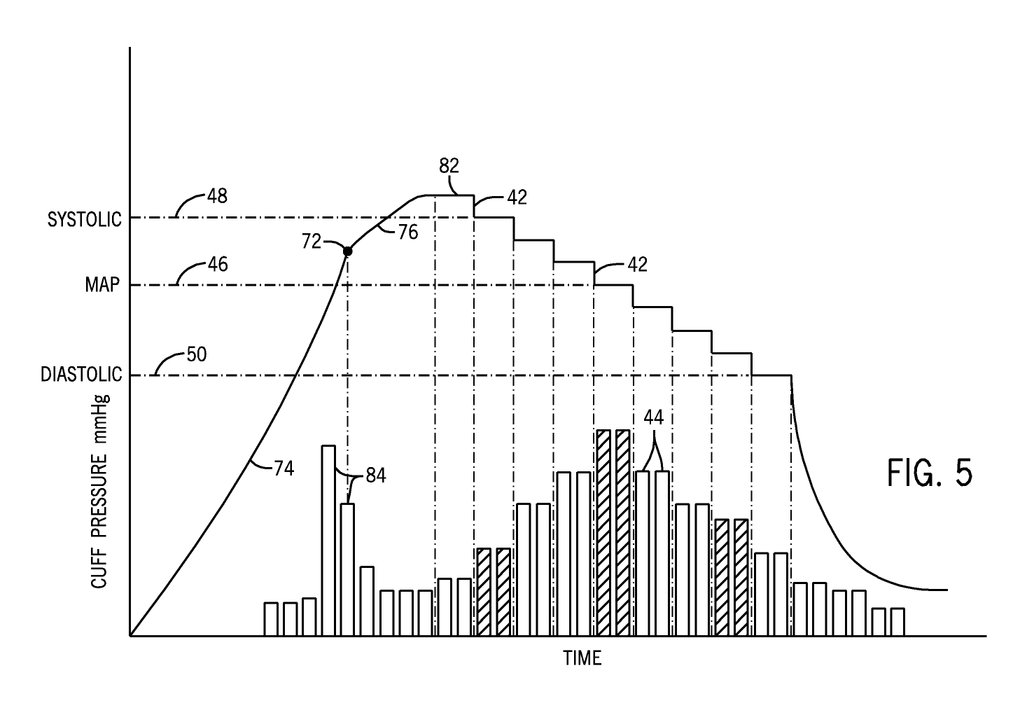

Referring now to FIG. 4, thereshown is an alternate embodiment of the NIBP monitoring system 10. In the embodiment shown in FIG. 4, the pulse monitor 52 of FIG. 1 is not required. In the embodiment of FIG. 4, the controller 22 again operates the air compressor 33 to inflate the blood pressure cuff at the rapid inflation rate to the first pressure 72, as is shown by the portion of the curve referred to by reference numeral 74 in FIG. 5. Once the blood pressure cuff has been inflated to the first pressure 72, the controller 22 again causes the air compressor 33 to inflate the blood pressure cuff at a measurement inflation rate, illustrated by the curve 76. During the inflation of the blood pressure cuff at the measurement inflation rate 76, the controller 22 monitors the signal from the pressure transducer 26 through the control line 30.

During the inflation of the blood pressure cuff at the measurement inflation rate shown by curve 76, the filtered oscillation signal from the blood pressure cuff will include a series of oscillation pulses 84. Each of the oscillation pulses 84 detected during the inflation period beneath the curve 76 generally correspond in intensity to the pulses 44 detected during deflation of the blood pressure cuff from the initial inflation pressure 82 for the same pressure levels. The pulses 84 detected during the inflation period beneath the curve 76 can be interpreted by the controller to estimate at least the systolic pressure for the patient. Since the inflation period shown by the portion of the curve 76 is much shorter than the deflection curve from the initial inflation pressure 82, the oscillation pulses detected during the portion of the curve 76 representing the measurement inflation rate are insufficient to calculate the final blood pressure of the patient. However, the oscillation pulses 84 detected during the inflation period can be utilized to estimate the systolic pressure for the patient.

Based upon the estimated systolic pressure, the controller once again calculates an initial inflation pressure 82 in the same manner as previously described. As illustrated in FIG. 5, the initial inflation pressure 82 is above the systolic pressure 48 for the patient. The initial inflation pressure 82 calculated during the inflation of the blood pressure cuff allows the NIBP monitoring system to more accurately initially inflate the blood pressure cuff as close as possible to the systolic pressure 48 to reduce the amount of time required to conduct the blood pressure measurement from the patient.

Since during the inflation of the blood pressure cuff only very small oscillation pulses will be detected from the pressure transducer 26 until the cuff pressure reaches the diastolic pressure 50, the controller rapidly inflates the blood pressure cuff at the rapid inflation rate shown by the portion of the curve 74 until the pressure reaches the first pressure 72. During the rapid inflation of the blood pressure cuff, the controller receives the oscillation pulses 84. The oscillation pulses 84 reach a maximum amplitude near the MAP for the patient. When the controller detects the decrease in the amplitude of the oscillation pulses, the controller signals the air compressor to decrease the rate of inflation, which takes place at the first pressure 72. Once the cuff pressure reaches the first pressure 72, the air compressor inflates the blood pressure cuff at the measurement inflation rate (curve 76) while the controller monitors for the oscillation pulses 84.

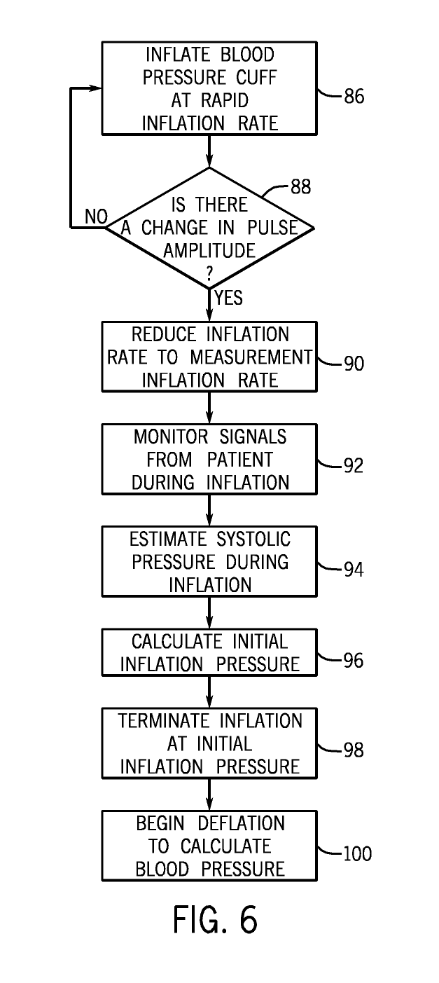

FIG. 6 illustrates a flow chart of the operational sequence of the NIBP monitoring system in accordance with one embodiment of the present disclosure. As illustrated in FIG. 6, the controller of the NIBP monitoring system 10 initially operates the inflate valve 36 to inflate the blood pressure cuff at the rapid inflation rate, as illustrated by step 86. In one embodiment, the inflate valve restricts the flow of pressurized air from a gas cylinder to control the inflation rate of the blood pressure cuff. In a second embodiment in which the source of pressurized air is from a variable air compressor, the controller controls the output of the compressor to provide the desired rapid inflation rate of the blood pressure cuff.

As the blood pressure cuff is being inflated, the controller monitors either the amplitude of the pulse signals 62 from the pulse monitor (FIG. 3) or the amplitude of the oscillation pulses 84 (FIG. 5) from the pressure transducer of the blood pressure cuff. When the controller detects the change in the amplitude of either of these two pulses, the controller signals either the air compressor or inflate valve to reduce the inflation rate, which occurs at the first pressure 72 in FIGS. 3 and 5.

Once the cuff pressure has reached the first pressure, the controller signals either the inflate valve 36 or the air compressor 33 to reduce the inflation rate to the measurement inflation rate, as shown in step 90. As previously described, the measurement inflation rate set in step 90 is less than the rapid inflation rate set in step 86. In the embodiment of FIGS. 3 and 5, the rapid inflation rate is shown by the curve 74 while the measurement inflation rate is shown by the curve 76.

During operation of the source of pressurized air to inflate the blood pressure cuff at the measurement inflation rate, the controller monitors signals from the patient during inflation, as shown in step 92. In the embodiment of FIGS. 1 and 3, the controller monitors a heart rate signal from a pulse monitor 52. In the embodiment shown in FIGS. 4 and 5, the controller monitors for the presence of oscillation pulses 84 during the inflation of the blood pressure cuff at the measurement inflation rate. In each embodiment, the controller generates an estimated systolic pressure for the patient based upon the signals received. Since the blood pressure cuff is inflated at the reduced measurement inflation rate shown in curve 76, the controller can analyze the signals received from the patient to generate the estimated systolic pressure, as shown in step 94.

Once the controller generates the estimated systolic pressure, the controller then calculates an initial inflation pressure 82, as best shown in step 96. As described, the initial inflation pressure is selected slightly above the estimated systolic pressure such that the blood pressure cuff is inflated above the systolic pressure for the patient. The selection of the initial inflation pressure 82 slightly above the predicted systolic pressure hopefully ensures that the blood pressure cuff will be inflated to an adequate pressure to ensure that the blood pressure measurement is taken from slightly above the systolic pressure for the patient.

Once the controller determines the initial inflation pressure in step 96, the controller terminates the inflation of the blood pressure cuff at the initial inflation pressure, as shown in step 98. Once the inflation has stopped, the controller begins to deflate the pressure within the blood pressure cuff in a series of pressure steps 42, as is conventional and illustrated by step 100. During the deflation of the blood pressure in the series of steps, the controller utilizes standard blood pressure monitoring algorithms to calculate the systolic, mean arterial pressure (MAP) and diastolic pressure for the patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of monitoring blood pressure in a patient, the method comprising the steps of:
positioning a blood pressure cuff on the patient, the blood pressure cuff being selectively inflatable to restrict blood flow past the blood pressure cuff;
inflating the blood pressure cuff at a rapid inflation rate to a first pressure;
continuing to inflate the blood pressure cuff above the first pressure at a measurement inflation rate, wherein the measurement inflation rate is less than the rapid inflation rate;
determining an initial inflation pressure for the patient during the inflation of the blood pressure cuff at the measurement inflation rate;
terminating the inflation of the blood pressure cuff at the initial inflation pressure;
decreasing the pressure in the blood pressure cuff from the initial inflation pressure while monitoring oscillation pulses from the blood pressure cuff; and
calculating the blood pressure for the patient based on the monitored oscillation pulses.

2. The method of claim 1 wherein the measurement inflation rate is sufficient to allow a determination of an estimated systolic pressure before the cuff pressure reaches the initial inflation pressure.

3. The method of claim 1 wherein the first pressure is below a predicted systolic pressure for the patient.

4. The method of claim 1 further comprising the steps of:
positioning an inflation valve between a source of pressurized air and the blood pressure cuff; and
operating the inflation valve to selectively inflate the blood pressure cuff at both the rapid inflation rate and the measurement inflation rate.

5. The method of claim 1 further comprising the steps of:
positioning a sensor of a pulse monitor on the patient;
monitoring for the presence of pulse signals from the pulse monitor during inflation of the blood pressure cuff at the measurement inflation rate; and
determining the initial inflation pressure based upon the pulse signals from the pulse monitor.

6. The method of claim 5 wherein the pulse monitor is an SpO₂ monitor and the sensor is positioned distal to the blood pressure cuff.

7. The method of claim 1 further comprising the steps of:
monitoring for the presence of oscillation pulses from the blood pressure cuff during inflation of the blood pressure cuff at the measurement inflation rate; and
determining the initial inflation pressure based on the oscillation pulses detected during the inflation of the blood pressure cuff at the measurement inflation rate.

8. The method of claim 7 wherein the first pressure is below a predicted systolic pressure such that the blood pressure cuff is inflated at the measurement inflation rate from the first pressure to the initial inflation pressure.

9. The method of claim 7 further comprising the steps of:
determining an estimated systolic pressure based on the oscillometric pulses detected during the inflation of the blood pressure cuff at the measurement inflation rate; and
determining the initial inflation pressure based upon the estimated systolic pressure.

10. A method of calculating an initial inflation pressure for a blood pressure cuff, comprising the steps of:
inflating the blood pressure cuff while positioned on the patient at a rapid inflation rate to a first pressure;
continuing to inflate the blood pressure cuff above the first pressure at a measurement inflation rate, wherein the measurement inflation rate is less than the rapid inflation rate; and
determining an initial inflation pressure for the patient based on a patient signal received during the inflation of the blood pressure cuff at the measurement inflation rate.

11. The method of claim 10 wherein the measurement inflation rate is sufficient to allow the determination of an estimated systolic pressure before the cuff pressure reaches the initial inflation pressure.

12. The method of claim 10 further comprising the steps of:
providing an air compressor to supply a source of pressurized gas to the blood pressure cuff; and
operating the air compressor to selectively inflate the blood pressure cuff at both the rapid inflation rate and the measurement inflation rate.

13. The method of claim 10 further comprising the steps of:
positioning an inflation valve between a source of pressurized air and the blood pressure cuff; and
operating the inflation valve to selectively inflate the blood pressure cuff at both the rapid inflation rate and the measurement inflation rate.

14. The method of claim 10 further comprising the steps of:
positioning a sensor of a pulse monitor on the patient;
monitoring for the presence of pulse signals from the pulse monitor during inflation of the blood pressure cuff at the measurement inflation rate; and
determining the initial inflation pressure based upon the pulse signals from the pulse monitor.

15. The method of claim 14 wherein the pulse monitor is an SpO₂ monitor and the sensor is positioned distal to the blood pressure cuff.

16. The method of claim 10 further comprising the steps of:
monitoring for the presence of oscillation pulses from the blood pressure cuff during inflation of the blood pressure cuff at the measurement inflation rate; and
determining the initial inflation pressure based on the oscillation pulses detected during the inflation of the blood pressure cuff at the measurement inflation rate.

17. The method of claim 16 further comprising the steps of:
determining an estimated systolic pressure for the patient based upon the oscillation pulses detected during the inflation of the blood pressure cuff at the measurement inflation rate; and
determining the initial inflation pressure based upon the estimated systolic pressure.

18. A system for non-invasively estimating a blood pressure of a patient, comprising:
a blood pressure cuff;
a variable source of pressurized air;
a controller coupled to the variable source of pressurized air, wherein the controller is configured to inflate the blood pressure cuff at a rapid inflation rate to a first pressure and to inflate the blood pressure cuff from the first pressure at a measurement inflation rate while calculating an initial inflation pressure during the inflation of the blood pressure cuff at the measurement inflation rate.

19. The system of claim 18 further comprising:
a pulse monitor having a sensor positioned on the patient to detect pulse signals from the patient,
wherein the controller determines the initial inflation pressure based upon the pulse signals detected from the patient.

20. The system of claim 18 wherein the blood pressure cuff includes a transducer configured to acquire a plurality of oscillation pulses during the inflation of the blood pressure cuff at the measurement inflation rate, wherein the controller determines the initial inflation pressure based upon the oscillation pulses detected during inflation at the measurement inflation rate.
